(19) United States
(12) Patent Application Publication
(10) Pub. No.: US 2022/0241376 A1
(43) Pub. Date: Aug. 4, 2022

(54) METHOD FOR DECREASING ADVERSE-EFFECTS OF INTERFERON

(71) Applicant: ENYO PHARMA, LYON (FR)

(72) Inventors: JACKY VONDERSCHER, SENOUILLAC (FR); ELISE ROY, LES FINS (FR); RAPHAËL DARTEIL, LYON (FR); PIETRO SCALFARO, FIESCH (CH)

(21) Appl. No.: 17/627,698

(22) PCT Filed: Jul. 17, 2020
(86) PCT No.: PCT/EP2020/070240

§ 371 Date: Jan. 17, 2022

(30) Foreign Application Priority Data
Jul. 18, 2019 (EP) 19186949.4

(51) Int. Cl. A61K 38/21; A61P 31/12; A61K 47/60; A61K 31/496; A61K 31/575; A61K 31/46

(52) U.S. Cl. CPC A61K 38/21; A61P 31/12; A61K 31/46; A61K 31/496; A61K 31/575; A61K 47/60

(57) ABSTRACT

The present invention relates to a method for decreasing adverse-effects of interferon and to new compositions and methods of treatment.

### FIELD OF THE INVENTION

The present invention relates to the field of medicine, especially to the use of interferon for the treatment of diseases or disorders.

### BACKGROUND OF THE INVENTION

Interferons (IFN) are a class of cytokines which are released by cells in response to the presence of several pathogens such as viruses, bacteria and parasites but also of tumor cells.

IFNs have been developed and marketed as drug therapies under different forms: wildtype cytokine or pegylated forms. In addition, variant of IFNs and controlled-release dosage form of IFNs are under development.

IFNs are used in therapy in various therapeutic area. IFNs are used for treating viral infection, particularly chronic viral infection, such as HBV (hepatitis B virus), HCV (hepatitis C virus), herpes virus, and papillomavirus (HPV). In addition, they are used for treating cancer, especially against hematopoietic cancers such as multiple myeloma, lymphoma and leukemia, or against solid tumors such as malignant melanoma, renal cell carcinoma and osteosarcoma. IFNα2a is used to treat viral infections, IFN-β1a and IFN-β1b are used to treat and control multiple sclerosis. IFN-γ is used for the treatment of chronic granulomatous disease, an immune disease.

However, it is also well-known that the treatment with interferons are often associated with adverse effects called “flu-like syndrome” or “influenza-like illness”, the said effects including fever, muscle pain, headache and fatigue. For example, the treatments with IFN (i.e., IFN-α, IFN-β and IFN-γ) are associated with these adverse effects with a very significant occurrence, in particular greater than 25% of patients and rather in about 50% of patients or more.

The adverse effects are a problem when prolonged therapy with IFNs is necessary. It has been even reported that a significant number of patients stopped prematurely the therapy due to these adverse effects. Similarly, due to the toxicity of high dose therapy with IFNs, studies have been performed with lower doses but the therapeutic efficiency was lost or significantly decreased. Accordingly, the adverse effects prompted researchers to find new therapy of diseases for which the IFN therapy has been demonstrated to be efficient. In addition, these adverse effects have greatly hindered the further development of IFN-based clinical treatments.

Therefore, there is a strong need of new therapeutic solutions for decreasing the adverse effects associated with IFNs treatment. Thereby, IFN therapy with high dose can be contemplated, its development for new therapeutic indications can be promoted, and the current treatments can be better-tolerated by the patients.

### SUMMARY OF THE INVENTION

The present invention relies on the discovery of the surprising capacity of an FXR agonist to decrease adverse effects of IFN therapy, in particular the flu-like syndrome. Accordingly, the FXR agonist increases the tolerance of a subject to the treatment with IFN.

Accordingly, the present invention relates to an FXR agonist for use for decreasing adverse effects resulting from a treatment with an interferon. It also relates to a pharmaceutical composition comprising an FXR agonist for use for decreasing adverse effects resulting from a treatment with an interferon. It further relates to the use of an FXR agonist for the manufacture of a medicament for use for decreasing adverse effects resulting from a treatment with an interferon. It relates to a method for decreasing the adverse effect of IFN therapy in a subject having a treatment with IFN, comprising administering an efficient amount of an FXR agonist and administering a therapeutically effective amount of IFN to said subject, thereby decreasing the adverse effects resulting from a treatment with the IFN.

In one aspect, the interferon is selected from the group consisting of IFN-α, IFN-β, IFN-γ, IFN-λ and a pegylated form thereof, and more particularly from the group consisting of IFN-α1a, IFN-α1b, IFN-α2a, IFN-α2b, IFN-β1a, IFN-β1b, IFN-γ1b, IFN-λ1a and a pegylated form thereof. In a particular aspect, the interferon is IFN-α2 or a pegylated form thereof, especially IFN-α2a, IFN-α2b or a pegylated form thereof. In a specific aspect, the interferon is IFN-α2a or a pegylated form thereof.

In one aspect, the FXR agonist is selected from the group consisting of FXR agonist disclosed in Table 1. In a specific aspect, the FXR agonist is EYP001. For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome.

In one aspect, the adverse effects are the flu-like syndrome, especially fever, weakness, muscle pain, headache, back or leg pain, bones or muscles aches, myalgia, and fatigue.

In another aspect, the present invention also relates to a pharmaceutical composition or a kit as a combined preparation for simultaneous, separate or sequential use, said pharmaceutical composition or kit comprising an IFN and an FXR agonist, wherein the IFN selected from the group consisting of IFN-α1a, IFN-α1b, and a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof. This pharmaceutical composition or kit is for use for the treatment of hepatitis B virus infection. In addition, the present invention relates to the use of this pharmaceutical composition or kit for the manufacture of a medicament for the treatment of hepatitis B virus infection. The present invention further relates to a method for treating hepatitis B virus infection in a subject, comprising administering a therapeutic effective amount of this pharmaceutical composition or comprising administering a therapeutic effective amount of an IFN as defined above and a therapeutic effective amount of an FXR agonist, thereby decreasing the adverse effects resulting from a treatment with the IFN. The FXR agonist can be selected from the group consisting of FXR agonist disclosed in Table 1. In a specific aspect, the FXR agonist is EYP001. For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome. In one aspect, the FXR agonist is administered at a therapeutic amount effective for decreasing the adverse effect of the IFN, especially the flu-like syndrome, and for decreasing the replication of hepatitis B virus infection.

In another aspect, the present invention also relates to a pharmaceutical composition or a kit as a combined preparation for simultaneous, separate or sequential use, said pharmaceutical composition or kit comprising an IFN-α and an FXR agonist for use for treating a disease selected from the group consisting of an infection by a virus chosen among hepatitis C virus (HCV), hepatitis D virus (HDV), herpes simplex virus (HSV), papillomavirus (HPV) (e.g., condylomata acuminate), varicella-zoster virus, cytomegalovirus (CMV) and rhinoviruses; a cancer, particularly a solid cancer or a hematopoietic cancer, preferably chosen among AIDS-related Kaposi's sarcoma, leukemia such as hairy-cell leukemia, chronic myeloid leukemia and non-Hodgkin's leukemia, lymphoma such as follicular lymphoma, cutaneous T-cell lymphoma and adult T-cell leukemia-lymphoma, carcinoid tumors, melanoma, multiple myeloma, renal cell carcinoma and neuroendocrine tumors; and other diseases such as age-related macular degeneration, angiomatous disease, Behçet's syndrome, thrombocythemia, polycythemia vera, agnogenic myeloid metaplasia, Churg-Strauss syndrome, inflammatory bowel disease and mycobacterial infection. In addition, the present invention relates to the use of this pharmaceutical composition or kit for the manufacture of a medicament for the treatment of a disease as defined above. The present invention further relates to a method for treating a disease as defined above in a subject, comprising administering a therapeutic effective amount of this pharmaceutical composition or comprising administering a therapeutic effective amount of an IFN-α and a therapeutic effective amount of an FXR agonist, thereby decreasing the adverse effects resulting from a treatment with the IFN. The FXR agonist can be selected from the group consisting of FXR agonist disclosed in Table 1. The IFN-α can be IFN-α1 or IFN-α2 or a pegylated form thereof, preferably selected from the group consisting of IFN-α1a, IFN-α1b, IFN-α2a and IFN-α2b or a pegylated form thereof.

In a specific aspect, the interferon is IFN-α2a or a pegylated form thereof. The FXR agonist can be selected from the group consisting of FXR agonist disclosed in Table 1. In a specific aspect, the FXR agonist is EYP001. For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome. In one aspect, the FXR agonist is administered at a therapeutic amount effective for decreasing the adverse effect of the IFN, especially the flu-like syndrome, and for having a therapeutic effect on one of the diseases as defined above.

In another aspect, the present invention also relates to a pharmaceutical composition or a kit as a combined preparation for simultaneous, separate or sequential use, said pharmaceutical composition or kit comprising an IFN-β and an FXR agonist for use for treating a disease selected from the group consisting of multiple sclerosis, Guillain-Barré syndrome, rheumatoid arthritis and a cancer, particularly a solid cancer or a hematopoietic cancer. In addition, the present invention relates to the use of this pharmaceutical composition or kit for the manufacture of a medicament for the treatment of a disease as defined above. The present invention further relates to a method for treating a disease as defined above in a subject, comprising administering a therapeutic effective amount of this pharmaceutical composition or comprising administering a therapeutic effective amount of an IFN-β and a therapeutic effective amount of an FXR agonist, thereby decreasing the adverse effects resulting from a treatment with the IFN. The IFN-β is preferably IFN-β1 or a pegylated form thereof, more preferably selected from the group consisting of IFN-β1a and IFN-β1b or a pegylated form thereof. The FXR agonist can be selected from the group consisting of FXR agonist disclosed in Table 1. In a specific aspect, the FXR agonist is EYP001.

For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome. In one aspect, the FXR agonist is administered at a therapeutic amount effective for decreasing the adverse effect of the IFN, especially the flu-like syndrome, and for having a therapeutic effect on one of the diseases as defined above.

In another aspect, the present invention also relates to a pharmaceutical composition or a kit as a combined preparation for simultaneous, separate or sequential use, said pharmaceutical composition or kit comprising an IFN-γ and an FXR agonist for use for treating a disease selected from the group consisting of bacterial infections, in particular mycobacterial infections, fibrosis such as cryptogenic fibrosing alveolitis, leishmaniasis, osteoporosis and a cancer, particularly a solid cancer or a hematopoietic cancer. In addition, the present invention relates to the use of this pharmaceutical composition or kit for the manufacture of a medicament for the treatment of a disease as defined above. The present invention further relates to a method for treating a disease as defined above in a subject, comprising administering a therapeutic effective amount of this pharmaceutical composition or comprising administering a therapeutic effective amount of an IFN-γ and a therapeutic effective amount of an FXR agonist, thereby decreasing the adverse effects resulting from a treatment with the IFN. The FXR agonist can be selected from the group consisting of FXR agonist disclosed in Table 1. In a specific aspect, the FXR agonist is EYP001.

For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome. In one aspect, the FXR agonist is administered at a therapeutic amount effective for decreasing the adverse effect of the IFN, especially the flu-like syndrome, and for having a therapeutic effect on one of the diseases as defined above.

In another aspect, the present invention also relates to a pharmaceutical composition or a kit as a combined preparation for simultaneous, separate or sequential use, said pharmaceutical composition or kit comprising an IFN-λ and an FXR agonist for use for treating a disease selected from the group consisting of fibrosis and a hepatitis D virus infection. In addition, the present invention relates to the use of this pharmaceutical composition or kit for the manufacture of a medicament for the treatment of a disease as defined above. The present invention further relates to a method for treating a disease as defined above in a subject, comprising administering a therapeutic effective amount of this pharmaceutical composition or comprising administering a therapeutic effective amount of an IFN-λ and a therapeutic effective amount of an FXR agonist, thereby decreasing the adverse effects resulting from a treatment with the IFN. The FXR agonist can be selected from the group consisting of FXR agonist disclosed in Table 1. In a specific aspect, the FXR agonist is EYP001. For instance, the FXR agonist can be administered once a day. It can also be administered twice a day. More particularly, the FXR agonist is administered as long as the treatment with IFN is carried out. In particular, the FXR agonist is administered at a therapeutic amount effective for decreasing the adverse effect of the IFN, especially the flu-like syndrome. In one aspect, the FXR agonist is administered at a therapeutic amount effective for decreasing the adverse effect of the IFN, especially the flu-like syndrome, and for having a therapeutic effect on one of the diseases as defined above.

### DETAILED DESCRIPTION OF THE INVENTION

The present invention relies on the discovery of the surprising capacity of an FXR agonist to decrease adverse effects of IFN therapy, in particular the flu-like syndrome. Experimental evidence has been provided with the FXR agonist EYP001 with side effects of pegylated IFNα, in particular on the flu-like syndrome. Firstly, the disease treated by the interferon has no impact on the side effects observed as a consequence of the treatment with interferon. The side effects are independent from the treated disease. Indeed, the flu-like syndrome has been observed during treatments of HBV infection by IFNα but it has also been observed during IFNα therapy of other diseases. Therefore, the present disclosure supports the effect of an FXR agonist on the side effects of IFN therapy whatever is the disease of the treated subject. Secondly, flu-like syndrome is not specific of IFN-α and is also observed with other interferons such as IFN-β (Takahashi, JMAJ, 2004, 47, 60-63; Patti et al, J Neurol, 2020, 267, 1812-1823) and IFN-γ (Vlachoyiannopoulos et al, Ann Rheum

Dis, 1996, 55, 761-768; Prescrire Int, 2006, 15, 179-180; Windbichler et al, Br J Cancer, 2000, 82, 1138-1144). Therefore, it is highly credible that an FXR agonist is able to decrease side effects associated with other interferons. Similarly, the flu-like syndrome is not specific of pegylated interferon. Then, an FXR agonist is able to decrease side effects of an interferon, pegylated or not. Accordingly, the application fully supports the use of EYP001 for decreasing side effects of an interferon. Finally, it is believed that the effect of EYP001 can also be obtained with alternative FXR agonists, especially the selective FXR agonist.

The present invention relates to an FXR agonist or a pharmaceutical composition comprising it for use for decreasing adverse effects resulting from a treatment with an interferon. Accordingly, the present invention also relates to an FXR agonist or a pharmaceutical composition comprising it for use for increasing the tolerance of a subject to a treatment with an interferon.

It further relates to the use of an FXR agonist or a pharmaceutical composition comprising it for the preparation of a medicament for decreasing adverse effects resulting from a treatment with an interferon.

In addition, it relates to a method for decreasing the adverse effects of resulting from a treatment with an interferon, comprising administering a therapeutically effective amount of an FXR agonist to the patient, thereby decreasing the adverse effects. More particularly, the method comprises administering a therapeutically effective amount of an interferon and a therapeutically effective amount of an FXR agonist. The therapeutically effective amount of an FXR agonist is the amount necessary for decreasing the adverse effects of interferon.

It finally relates to a kit comprising an interferon and an FXR agonist as a combined preparation for simultaneous, separate or sequential use for decreasing the adverse effects of interferon during a treatment with the interferon.

More specifically, the adverse effects of interferon are the flu-like syndrome. This syndrome includes: fever, weakness, muscle pain, headache, back or leg pain, bones or muscles aches, myalgia and fatigue. The FXR agonist decreases at least one aspect of the flu-like syndrome, for instance an aspect selected among fever, muscle pain, headache and fatigue. Preferably, the FXR agonist decreases several aspects of the flu-like syndrome, e.g., two or three.

By decreasing the adverse effects, it is intended that the adverse effects are decreased in the frequency of occurrence in a treated patient or in the population of treated patients and/or the adverse effects are decreased in their intensity and/or the appearance of the adverse effects is delayed. In particular, the decrease is of at least 10, 20, 30, 40 or 50%. The FXR agonist is to be administered in an amount necessary for decreasing the adverse effects of interferon during a treatment with the interferon. In a first aspect, the interferon is to be used with the recommended dosage for the therapeutic indication. Alternatively, the FXR agonist can be used so as higher dosage of interferon can be used without an increase of the adverse effects. For instance, an increase of 10, 20, 30, 40, 50, 60, 70, 80, 90, or 100% of the interferon dosage can be contemplated when used in combination with an FXR agonist.

The interferon can be for use for the treatment of a virus infection or a cancer. In one aspect, the virus infection is hepatitis B virus (HBV), hepatitis C virus (HCV), hepatitis D virus (HDV), herpes simplex virus (HSV), papillomavirus (HPV) (e.g., condylomata acuminate), varicella-zoster virus, cytomegalovirus (CMV) or rhinoviruses. In one particular embodiment, the virus infection is not an infection by the hepatitis B virus. In another aspect, the cancer is a solid cancer or an hematopoietic cancer, preferably AIDS-related Kaposi's sarcoma, leukemia such as hairy-cell leukemia, chronic myeloid leukemia and non-Hodgkin's leukemia, lymphoma such as follicular lymphoma, cutaneous T-cell lymphoma and adult T-cell leukemia-lymphoma, carcinoid tumors, melanoma, multiple myeloma, renal cell carcinoma and neuroendocrine tumors. In a further aspect, the interferon is for use for treating other diseases selected from the group consisting of multiple sclerosis, Guillain-Barré syndrome, rheumatoid arthritis, age-related macular degeneration, angiomatous disease, Behçet's syndrome, thrombocythemia, polycythemia vera, agnogenic myeloid metaplasia, Churg-Strauss syndrome, inflammatory bowel disease, bacterial infection such as mycobacterial infection, fibrosis, leishmaniasis, and osteoporosis.

Definition

The term “FXR” refers to the farnesoid X receptor, which is a nuclear receptor that is activated by supraphysiological levels of farnesol (Forman et al., Cell, 1995,81,687-693). FXR, is also known as NR1H4, retinoid X receptor-interacting protein 14 (RIP14) and bile acid receptor (BAR). Containing a conserved DNA-binding domain (DBD) and a C-terminal ligand-binding domain (LBD), FXR binds to and becomes activated by a variety of naturally occurring bile acids (BAs), including the primary bile acid chenodeoxycholic acid (CDCA) and its taurine and glycine conjugates. Upon activation, the FXR-RXR heterodimer binds the promoter region of target genes and regulates the expression of several genes involved in bile acid homeostasis. Hepatic FXR target genes fall into two main groups. The first group functions to decrease hepatic bile acids concentrations by increasing export and decreasing their synthesis. The second group of FXR target genes such as the phospholipid transport protein PLTP and apolipoproteins modulates lipoprotein levels in the serum and decreases plasma triglyceride concentration. For a more detailed list of FXR-regulated genes, see, e.g., WO 03/016288, pages 22-23. U.S. Pat. No. 6,005,086 discloses the nucleic acid sequence coding for a mammalian FXR protein. The human polypeptide sequences for FXR are deposited in nucleotide and protein databases under accession numbers NM_005123, Q96RI1, NP_005114 AAM53551, AAM53550, AAK60271.

In this specification, the term “FXR agonist” has its general meaning in the art and refers in particular to compounds that function by targeting and binding the farnesoid X receptor (FXR) and which activate FXR by at least 40% above background in the assay described in Maloney et al. (J. Med. Chem. 2000, 43:2971-2974).

In some embodiments, the FXR agonist of the invention is a selective FXR agonist. As used herein, the term “selective FXR agonist” refers to an FXR agonist that exhibits no significant cross-reactivity to one or more, ideally substantially all, of a panel of nuclear receptors consisting of LXRα, LXRβ, PPARα, PPARγ, PPARδ, RXRα, RARγ, VDR, PXR, ERα, ERβ, GR, AR, MR and PR. Methods of determining significant cross-reactivity are described in J. Med. Chem. 2009, 52, 904-907.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease. As used herein, the terms “subject”, “individual” or “patient” are interchangeable and refer to a human, including adult, child, newborn and human at the prenatal stage. Alternatively, animals, in particular pets or farm or zoo animals, can also be considered as “subject”, “individual” or “patient”.

The terms “quantity,” “amount,” and “dose” are used interchangeably herein and may refer to an absolute quantification of a molecule.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “therapeutically effective amount” refers to a quantity of an active ingredient or of a pharmaceutical composition which prevents, removes or reduces the deleterious effects of the disease, particularly infectious disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

As used herein, the term “pegylated form” refers to a pegylated interferon.

Interferon

The interferon can be any IFN.

In one aspect, IFN is selected from type I IFN, type II IFN and type III IFN.

Type I IFNs bind the IFN-α/β receptor. Type I IFNs includes IFN-α (alpha), IFN-β (beta), IFN-κ (kappa), IFN-δ (delta), IFN-ε (epsilon), IFN-τ (tau), IFN-ω (omega) and IFN-ζ (zeta). Preferably, Type I IFN is IFN-α or IFN-μ. IFN-α contains 13 subtypes (indicated IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21). These subtypes can be divided into various sub-subtypes such as IFN-α1a, IFN-α1b, IFN-α2a, IFN-α2b. Similarly, IFN-β contains several subtypes such as IFN-β1 and IFN-β3, divided in sub-subtypes such as IFN-β1a, IFN-β1b, etc . . .

Type II IFN includes IFN-γ. In particular, IFN-γ can be IFN-γ1, especially IFN-γ1b.

Type III IFNs include IFN-λ. It includes non-exhaustively IFN-λ1, IFN-λ2, IFN-λ3 and IFN-λ4.

IFN encompasses salts, functional derivatives, variants, muteins, fused proteins, analogs and active fragments thereof, said IFN having the same functional effect than the wildtype IFN. Alternatively, IFN can be a derivatized form of IFN, in particular for increasing its half-life. Accordingly, IFN can be derivatized with a water-soluble polymer such as polyethylene glycol, i.e. pegylated IFN. Such pegylated IFNs are described in U.S. Pat. Nos. 5,382,657; 5,951,974; and 5,981,709 (the disclosure thereof being incorporated by reference).

Variants of IFN are well-known in the art, for instance for IFN-α, see W02013107791, Piehler et al (2000, J Biol Chem, 275, 40425-33), WO2010030671, WO2008124086, WO2015007520, WO2013059885, for IFN-γ, see WO18077893, WO18064574.

In one aspect, IFN is a pegylated IFN, more particularly a pegylated type I IFN, especially a pegylated IFN-α such as a pegylated IFN-α2 including a pegylated IFN-α2a or a pegylated IFN-α2b; a pegylated IFN-β (e.g., IFN-β1a or IFN-β1b) or a pegylated IFN-γ.

In one aspect, IFN is selected from the group consisting of consensus IFN-α (e.g., INFERGEN®, Locteron®), IFN-α1b (e.g., HAPGEN®), IFN-α2a (Roferon-A®, MOR-22, Inter 2A, Inmutag, Inferon), a pegylated IFN-α2a (e.g., PEGASYS®, YPEG-IFNα-2a, PEG-INTRON®, Pegaferon), IFN-α2b (e.g., INTRON A®, Alfarona, Bioferon, Inter 2B, citpheron, Zavinex, Ganapar, etc . . . ), a pegylated IFN-α2b (e.g., Pegintron®, Albuferon, AOP2014/P1101, Algeron, Pai Ge Bin), IFN-α2c (e.g. Berofor Alpha), IFN-β1a (e.g., REBIF®, AVONEX®), a pegylated IFN-β1a (e.g. Plegridy), IFN-β1b (e.g., Betaseron®), IFN-γ (e.g., Ingaron), a pegylated IFN-γ (e.g., Ingaron), and IFN-like protein (e.g., Novaferon, HSA-IFN-α2a fusion protein, HSA-IFN-α2b fusion protein).

IFN can be administered daily, weekly or 2, 3, 4, 5, or 6 times weekly. The treatment period is generally long, for instance from 2 weeks to several months. For instance, the period is from 3-4 months up to 24 months. The dosage can vary from 1 million units to 20 million units, for instance 2, 3, 4, 5, 6, 7, 8, 9, 10, 11, 12, 13, 14, 15, 16, 17, 18 or 19 million units.

IFN can be administered by subcutaneous, intramuscular, intravenous, transdermal, or intratumoral administration, preferably for subcutaneous or intramuscular administration. In a particular aspect, the IFN is IFNα2a, IFNα2b or a pegylated form thereof and is administered subcutaneously once a week, for instance at a dosage varying from 1 μg to 500 μg, preferably from 10 μg to 500 μg, more preferably from 100 μg to 250 μg, such as 100, 110, 120, 130, 140, 150, 160, 170, 180, 190 or 200 μg, and during from 2-4 months up to 24 months. In a very specific aspect, the treatment lasts from 12 to 52 weeks, preferably from 45 to 52 weeks, for instance 48 weeks. In a more specific aspect, the IFN is IFNα2a or a pegylated form thereof

FXR Agonist

FXR agonists are well known to the skilled person.

For example, the skilled person may easily identify FXR agonist from the following publications (the disclosure of which being incorporated herein by reference):
Abenavoli L, et al. Pharmaceuticals (Basel). 2018 Oct. 11;11(4). pii: E104. doi: 10.3390/ph11040104. Review.
Adorini L, et al. Drug Discov Today. 2012 September;17(17-18):988-97. doi: 10.1016/j.drudis.2012.05.012. Epub 2012 May 29. Review.
Akwabi-Ameyaw A, et al. Bioorg Med Chem Lett. 2009 Aug. 15;19(16):4733-9. doi: 10.1016/j.bmcl.2009.06.062. Epub 2009 Jun. 21.
Akwabi-Ameyaw A, et al. Bioorg Med Chem Lett. 2008 Aug. 1;18(15):4339-43. doi: 10.1016/j.bmcl.2008.06.073. Epub 2008 Jun. 28.
Akwabi-Ameyaw A, et al. Bioorg Med Chem Lett. 2011 Oct. 15;21(20):6154-60. doi: 10.1016/j.bmcl.2011.08.034. Epub 2011 Aug. 11.
Baghdasaryan A, et al. Hepatology. 2011 October;54(4):1303-12. doi: 10.1002/hep.24537.
Bass J Y, et al. Bioorg Med Chem Lett. 2009 Jun. 1;19(11):2969-73. doi: 10.1016/j.bmcl.2009.04.047. Epub 2009 Apr. 18.
Bass J Y, et al. Bioorg Med Chem Lett. 2011 Feb. 15;21(4):1206-13. doi: 10.1016/j.bmcl.2010.12.089. Epub 2010 Dec. 23.
Buijsman et al., Curr. Med. Chem. 2005, 12, 1017
Carino et al, Sci Rep. 2017 Feb. 16;7:42801. doi: 10.1038/srep42801.
Chiang P C, et al. J Pharm Sci. 2011 November;100(11):4722-33. doi: 10.1002/jps.22664. Epub 2011 Jun. 9.
Crawley, Expert Opin. Ther. Pat. 2010, 20, 1047
Feng S, et al. Bioorg Med Chem Lett. 2009 May 1;19(9):2595-8. doi: 10.1016/j.bmcl.2009.03.008. Epub 2009 Mar. 9.
Festa et al, Front Pharmacol. 2017 Mar. 30;8:162. doi: 10.3389/fphar.2017.00162. eCollection 2017.
Finamore et al, Sci Rep. 2016 Jul. 6;6:29320. doi: 10.1038/srep29320.
Flatt B, et al. J Med Chem. 2009 Feb. 26;52(4):904-7. doi: 10.1021/jm8014124.
Gege et al, Curr Top Med Chem. 2014;14(19):2143-58.
Gege et al, Handbook of Experimental Pharmacology, doi: 10.1007/164_2019_232.
Genin et al, J Med Chem. 2015 Dec. 24;58(24):9768-72. doi: 10.1021/acs.jmedchem.5b01161. Epub 2015 Dec. 2.
Ghebremariam Y T, et al. PLoS One. 2013 Apr. 4;8(4):e60653. doi: 10.1371/journal.pone.0060653. Print 2013.
Gioiello A, et al. Bioorg Med Chem. 2011 Apr. 15;19(8):2650-8. doi: 10.1016/j.bmc.2011.03.004. Epub 2011 Mar. 10.
Hoekstra M, et al. Mol Cell Endocrinol. 2012 Oct. 15;362(1-2):69-75. doi: 10.1016/j.mce.2012.05.010. Epub 2012 May 27.
Iguchi Y, et al. Steroids. 2010 January;75(1):95-100. doi: 10.1016/j.steroids.2009.11.002. Epub 2009 Nov. 12.
Kinzel et al, Bioorg Med Chem Lett. 2016 Aug. 1;26(15):3746-53. doi: 10.1016/j.bmcl.2016.05.070. Epub 2016 May 24.
Lin H R. Bioorg Med Chem Lett. 2012 Jul. 15;22(14):4787-92. doi: 10.1016/j.bmcl.2012.05.057. Epub 2012 May 23.
Lundquist J T, et al. J Med Chem. 2010 Feb. 25;53(4):1774-87. doi: 10.1021/jm901650u.
Ma Y, et al. Pharm Res. 2013 May;30(5):1447-57. doi: 10.1007/511095-013-0986-7. Epub 2013 Feb. 1.
Marinozzi M, et al. Bioorg Med Chem. 2013 Jul. 1;21(13):3780-9. doi: 10.1016/j.bmc.2013.04.038. Epub 2013 Apr. 23.
Massafra et al. Pharmacol Ther. 2018 November;191:162-177. doi: 10.1016/j.pharmthera.2018.06.009. Epub 2018 Jun. 20.
Misawa T, et al. Bioorg Med Chem Lett. 2012 Jun. 15;22(12):3962-6. doi: 10.1016/j.bmcl.2012.04.099. Epub 2012 Apr. 30.
Pellicciari et al, J Med Chem. 2016 Oct. 4.
Richter H G, et al. Bioorg Med Chem Lett. 2011 Feb. 15;21(4):1134-40. doi: 10.1016/j.bmcl.2010.12.123. Epub 2010 Dec. 31.
Rizzo G, et al. Mol Pharmacol. 2010 October;78(4):617-30. doi: 10.1124/mo1.110.064501. Epub 2010 Jul. 14.
Roda et al, J Pharmacol Exp Ther. 2014 July;350(1):56-68. doi: 10.1124/jpet.114.214650. Epub 2014 May 1.
Schuster D, et al. Bioorg Med Chem. 2011 Dec. 1;19(23):7168-80. doi: 10.1016/j.bmc.2011.09.056. Epub 2011 Oct. 4.
Schwabl et al, J Hepatol. 2017 April;66(4):724-733. doi: 10.1016/j.jhep.2016.12.005. Epub 2016 Dec. 18.
Samlley et al, Bioorg Med Chem Lett. 2015 Jan. 15;25(2):280-4. doi: 10.1016/j.bmcl.2014.11.050. Epub 2014 Nov. 26.
Sepe et al. Expert Opin Ther Pat. 2018 May;28(5):351-364. doi: 10.1080/13543776.2018.1459569. Epub 2018 Apr. 13. Review.
Sepe et al. Expert Opin Ther Pat. 2015;25(8):885-96. doi: 10.1517/13543776.2015.1045413. Review.
Soisson S M, et al. Proc Natl Acad Sci U S A. 2008 Apr. 8;105(14):5337-42. doi: 10.1073/pnas.0710981105. Epub 2008 Apr. 7.
Townsend S A, Newsome P N. Aliment Pharmacol Ther. 2017 September;46(5):494-507. doi: 10.1111/apt.14210. Epub 2017 Jul. 4.
Tully et al, J Med Chem. 2017 Dec. 28;60(24):9960-9973. doi: 10.1021/acs.jmedchem.7b00907. Epub 2017 Dec. 8.
Wang et al, J Am Soc Nephrol. 2018 January;29(1):118-137. doi: 10.1681/ASN.2017020222. Epub 2017 Oct. 31.
Wang et al, Bioorg Med Chem Lett. 2017 Aug. 1;27(15):3386-3390. doi: 10.1016/j.bmcl.2017.06.003. Epub 2017 Jun. 3.
Wang H, et al. Expert Opin Ther Pat. 2018 November;28(11):765-782. doi: 10.1080/13543776.2018.1527906. Epub 2018 Oct. 8. Review
Watanabe M, et al. J Biot Chem. 2011 Jul. 29;286(30):26913-20. doi: 10.1074/jbc.M111.248203. Epub 2011 Jun. 1.
Yu D, et al. Steroids. 2012 November;77(13):1335-8. doi: 10.1016/j.steroids.2012.09.002. Epub 2012 Sep. 21.
Zhang S, et al. J Hepatol. 2009 August;51(2):380-8. doi: 10.1016/j.jhep.2009.03.025. Epub 2009 May 18.

Typically, FXR agonists include the class of steroid FXR agonists and non-steroid FXR agonists.

In certain embodiments of the invention, the FXR agonist is selected from small molecule compounds which act as FXR modulators that have been disclosed in the following publications: EP1392714; EP1568706; JP2005281155; US20030203939; US2005080064; US2006128764; US20070015796; US20080038435; US20100184809; US20110105475; US6,984,560; WO2000037077; WO200040965; WO200076523; WO2003015771; WO2003015777; WO2003016280; WO2003016288; WO2003030612; WO2003016288; WO2003080803; WO2003090745; WO2004007521; WO2004048349; WO2004046162; WO2004048349; WO2005082925; WO2005092328; WO2005097097; WO2007076260; WO2007092751; WO2007140174; WO2007140183; WO2008002573; WO2008025539; WO2008025540; WO200802573; WO2008051942; WO2008073825; WO2008157270; WO2009005998; WO2009012125; WO2009027264; WO2009080555; WO2009127321; WO2009149795; WO2010028981; WO2010034649; WO2010034657; WO2017218330; WO2017218379; WO2017201155; WO2017201152; WO2017201150; WO2017189652; WO2017189651; WO2017189663; WO2017147137; WO2017147159; WO2017147174; WO2017145031; WO2017145040; WO2017145041; WO2017133521; WO2017129125; WO2017128896; WO2017118294; WO2017049172; WO2017049176; WO2017049173; WO2017049177; WO2016173397; WO2016173493; WO2016168553; WO2016161003; WO2016149111; WO2016131414; WO2016130809; WO2016097933; WO2016096115; WO2016096116; WO2016086115; WO2016073767; WO2015138986; WO2018152171; WO2018170165, WO2018170166, WO2018170173, WO2018170182, WO2018170167; WO2017078928; WO2014184271; WO2013007387; WO2012087519; WO2011020615; WO2010069604; WO2013037482; US2017275256; WO2005080064; WO2018190643; WO2018215070; WO2018215610; WO2018214959; WO2018081285; WO2018067704; WO2019007418; WO2018059314; WO2017218337; the disclosure of which being incorporated herein by reference.

In an aspect, the FXR agonist can be any FXR agonists disclosed in the following patent applications: WO2017/049172, WO2017/049176, WO2017/049173, WO2017/049177, WO2018/170165, WO2018/170166, WO2018/170173, WO2018/170182, and WO2018/170167.

Specific examples of FXR agonists include but are not limited to EYP001, GW4064 (as disclosed in PCT Publication No. WO 00/37077 or in US2007/0015796), 6-ethyl-chenodeoxycholic acids, especially 3α, 7α-dihydroxy 7α-dihydroxy-6α-ethyl-5β-cholan-24-oic acid, also referred to as INT-747; INT-777; 6-ethyl-ursodeoxycholic acids, INT-1103, UPF-987, WAY-362450, MFA-1, GW9662, T0901317, fexaramine, 3β-azido-6α-ethyl-7α-hydroxy-5β-cholan-24-oic acid, Tropifexor (LIN452), fexaramine-3 (Fex-3), BAR502, BAR704, PX20606, PX20350, 3α,7α,11β-Trihydroxy-6α-ethyl-5β-cholan-24-oic Acid (TC-100), 6-(4-{[5-Cyclopropyl-3-(2,6-dichlorophenyl)isoxazol-4-yl]methoxy}piperidin-1-yl)-1-methyl-1H-indole-3-carboxylic Acid, 3,6-dimethyl-1-(2-methylphenyl)-4-(4-phenoxyphenyl)-4,8-dihydro-1H-pyrazolo[3,4-e][1,4]thiazepin-7-one; obeticholic acid, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, GSK-8062 (CAS No. 943549-47-1). In some embodiments, the FXR agonist is selected from natural bile acids, preferably chenodeoxycholic acid [CDCA] or taurine- or glycine-conjugated CDCA [tauro-CDCA or glyco-CDCA] and synthetic derivatives of natural bile acids, preferably 6-Ethyl-CDCA or taurine- or glycine-conjugated 6-Ethyl-CDCA, natural non-steroidal agonists, preferably Diterpenoids such as Cafestol and Kahweol, or synthetic non-steroidal FXR agonists.

In some embodiments, the FXR agonist is selected from the group consisting of obeticholic acid (Intercept Pharma), cholic acid (CT-RS); GS-9674 (Cilofexor) (Phenex Pharmaceuticals AG), Tropifezor (LIN452) (Novartis Pharmaceuticals), EYP001, EDP-305, a steroidal non-carboxylic acid FXR agonist (Enanta Pharmaceuticals), Turofexorate Isopropyl (Pfizer), INT-767 (Intercept Pharmaceuticals), LY-2562175 (Lilly), AGN-242266 (former AKN-083, Allergan), EP-024297 (Enanta Pharmaceuticals), M-480 (Metacrine), MET-409 (Metacrine), RDX-023 (Ardelyx), GW6046, Cafestol, Fexaramine and the compound PXL007 (also named EYP001 or EYP001a) identified by the CAS No. 1192171-69-9 (described in WO 2009127321). In a particular embodiment, the FXR agonist is selected from the group consisting of INT-747, the compound identified by EDP-305 a steroidal non-carboxylic acid FXR agonist (Enanta Pharmaceuticals) and the compound identified by the CAS No. 1192171-69-9 (described in WO 2009127321).

In a particular aspect, the FXR agonist is selected from the group consisting of LIN452 (Tropifezor), GS-9674 (Cilofexor), LMB763 (Nidufexor), OCA (Ocaliva), EDP-305, TERN-001 and PXL007 (also named EYP001). In a particular aspect, the FXR agonist is selected from the group consisting of the compound disclosed in Table 1.

TABLE 1

| | |
|---|---|
| LJN452 (Tropifexor) Cas Number 1383816-29-2 2-(3-((5-cyclopropyl-3-(2- (trifluoromethoxy)phenyl) isoxazol-4-yl)methoxy)-8- azabicyclo[3.2.1]octan-8- yl)-4-fluorobenzo[d] thiazole-6-carboxylic acid | 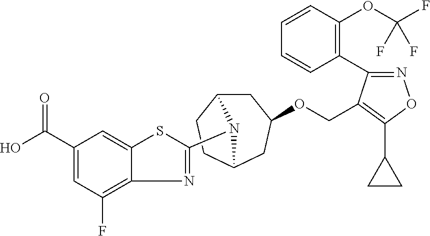 [SMILES: O=C(O)c1cc(F)c2nc(N3[C@@H]4CC[C@H]3C[C@@H](OCc3c(-c5ccccc5OC(F)(F)F)noc3C3CC3)C4)sc2c1] |
| LMB763 (Nidufexor) Cas Number 1773489-72-7 4-[(N-benzyl-8-chloro-1- methyl-1,4-dihydro[1] benzopyrano[4,3-c] pyrazole-3-carboxamido) methyl]benzoic acid | 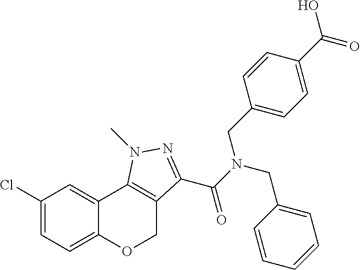 [SMILES: Cn1nc(C(=O)N(Cc2ccccc2)Cc2ccc(C(=O)O)cc2)c2c1-c1cc(Cl)ccc1OC2] |
| GS-9674 (Cilofexor) Cas Number 1418274-28-8 2-[3-[2-Chloro-4-[[5-cyclo- propyl-3-(2,6-dichlorophenyl)- 4-isoxazolyl]methoxy] phenyl]-3-hydroxy-1- azetidinyl]-4-pyridine- carboxylic acid | 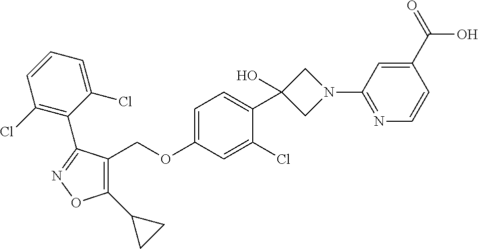 [SMILES: O=C(O)c1ccnc(N2CC(O)(c3ccc(OCc4c(-c5c(Cl)cccc5Cl)noc4C4CC4)cc3Cl)C2)c1] |
| PX-102 (PX-20606) Cas Number 1268244-85-4 4-(2-(2-Chloro-4-((5- cyclopropyl-3-(2,6- dichlorophenyl)isoxazol- 4-yl)methoxy)phenyl) cyclopropyl)benzoic acid | 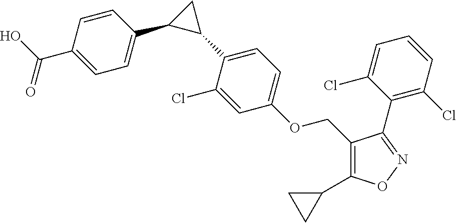 [SMILES: O=C(O)c1ccc([C@H]2C[C@@H]2c2ccc(OCc3c(-c4c(Cl)cccc4Cl)noc3C3CC3)cc2Cl)cc1] |
| PX-104 or Phenex 104 | enantiomer of PX-102 |
| OCA (Ocaliva or INT-747) Cas Number 459789-99-2 Cholan-24-oic acid, 6- ethyl-3,7-dihydroxy-, (3α,5β,6α,7α)- | 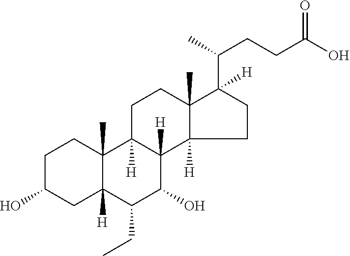 |
| EDP-305 Cas Number 1933507-63-1 Benzenesulfonamide, 4-(1,1- dimethylethyl)-N- [[[(3α,5β,6α,7α)-6-ethyl-3,7- dihydroxy-24-norcholan-23- yl]amino]carbonyl]- | 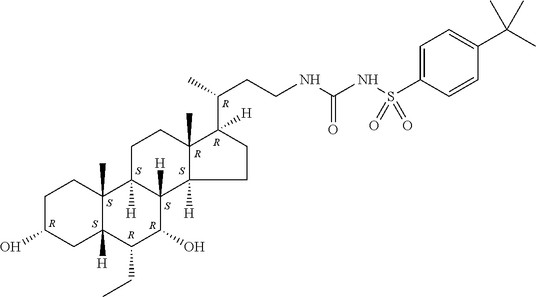 |
| TERN-101 (LY2562175) Cas Number 1103500-20-4 6-(4-{[5-Cyclopropyl-3-(2,6- dichlorophenyl)isoxazol-4- yl]methoxy}piperidin-1-yl)- 1-methyl-1H-indole-3- carboxylic acid | 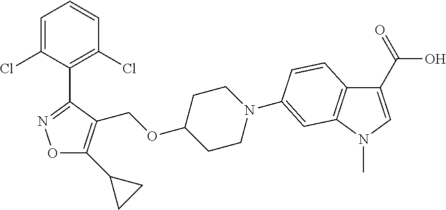 [SMILES: Cn1cc(C(=O)O)c2ccc(N3CCC(OCc4c(-c5c(Cl)cccc5Cl)noc4C4CC4)CC3)cc21] |
| MET409 | 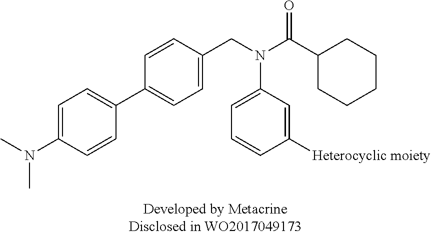 |
| GW4064 Cas Number 278779-30-9 3-[2-[2-Chloro-4-[[3-(2,6- dichlorophenyl)-5-(1- methylethyl)-4-isoxazolyl] methoxy]phenyl]ethenyl] benzoic acid | 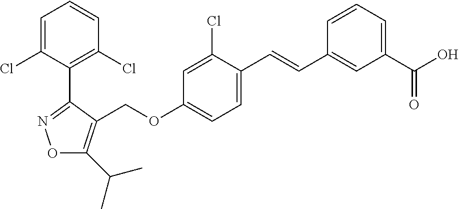 [SMILES: CC(C)c1onc(-c2c(Cl)cccc2Cl)c1COc1ccc(/C=C/c2cccc(C(=O)O)c2)c(Cl)c1] |
| WAY362450 (Turofexorate isopropyl or XL335 or FXR450) Cas Number 629664-81-9 3-(3,4-Difluoro-benzoyl)- 1,1-dimethylene-1,2,3,6- tetrahydro-azepino [4,5-b] indole-5-carboxylic acid isopropyl ester, 3-(3,4- Difluorobenzoyl)-1,2,3,6- tetrahydro-1,1-dimethyl- azepino[4,5-b]indole-5- carboxylic acid 1-methyl- ethyl ester, | 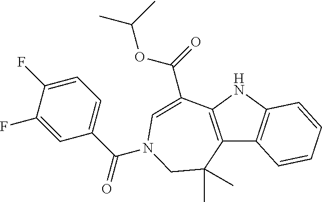 [SMILES: CC(C)OC(=O)C1=CN(C(=O)c2ccc(F)c(F)c2)CC(C)(C)c2c1[nH]c1ccccc21] |
| Fexaramine Cas Number 574013-66-4 3-[3-[(Cyclohexylcarbonyl) [[4′-(dimethylamino)[1,1′- biphenyl]-4-yl]methyl] amino]phenyl]-2-propenoic acid methyl ester | 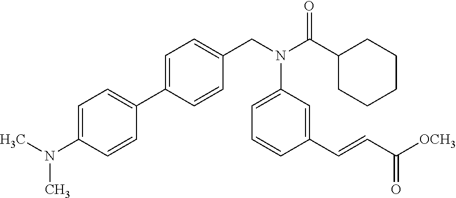 [SMILES: COC(=O)/C=C/c1cccc(N(Cc2ccc(-c3ccc(N(C)C)cc3)cc2)C(=O)C2CCCCC2)c1] |
| AGN242266 (AKN-083) | 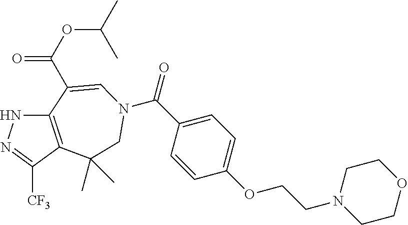 [SMILES: CC(C)OC(=O)C1=CN(C(=O)c2ccc(OCCN3CCOCC3)cc2)CC(C)(C)c2c(C(F)(F)F)n[nH]c21] |
| BAR502 Cas Number 1612191-86-2 6α-ethyl-3α, 7α-dihydroxy- 24-nor-5β-cholan-23-ol | 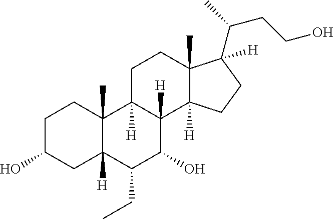 |
| EYP001 Cas Number 1192171-69-9 | 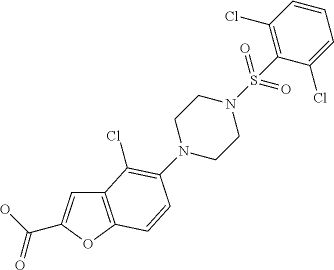 [SMILES: O=C(O)c1cc2c(Cl)c(N3CCN(S(=O)(=O)c4c(Cl)cccc4Cl)CC3)ccc2o1] |

and any pharmaceutically acceptable salt thereof.

In a preferred aspect of the invention, the FXR agonist is EYP001.

The FXR agonist can be administered with or without food (i.e., under fed conditions or under fasted conditions, respectively). It can be administered once, twice or three times a day, preferably once or twice, for example in the morning (e.g., between 6 and 10 am) or in the evening (e.g., 6 and 10 pm). In one aspect, the FXR agonist is administered once a day. In another aspect, the FXR agonist is administered twice a day. It is preferably administered every day. However, an administration every 2, 3, 4, 5, 6 or 7 days can also be contemplated. The daily dosage of the FXR agonist may be varied over a wide range from 0.01 to 1,000 mg per adult per day, especially from 1 to 1,000 mg per adult per day, preferably from 50 to 800 mg per adult per day, more preferably from 100 to 600 mg per adult per day, still more preferably from 150 to 400 mg per adult per day or from 200 to 400 mg per adult per day. Preferably, the compositions 5, 10, 15, 25, 50, 75, 100, 150, 200, 300, 400 or 500 mg of the FXR agonist. A medicament typically contains from about 0.05 mg to about 500 mg of FXR agonist, preferably from about 5 mg to about 500 mg of FXR agonist a day, preferably from 50 mg to about 500 mg of FXR agonist. The FXR agonist can be administered by oral, sublingual, subcutaneous, intramuscular, intravenous, transdermal, local or rectal administration, preferably for oral administration.

Pharmaceutical Compositions and Kits

One aspect of the disclosure relates to a pharmaceutical composition comprising an IFN and an FXR agonist.

In particular, the IFN is an IFN-α, preferably IFN-α1 or IFN-α2, such as IFN-α1a, IFN-α1b, IFN-α2a, and IFN-α2b or a pegylated form thereof. Alternatively, the IFN is an IFN-β, preferably IFN-β1 such as IFN-β1a and IFN-β1b or a pegylated form thereof. The IFN can also be IFN-γ1, especially IFN-γ1b, or a pegylated form thereof. The IFN can be an IFN-λ or IFN-λ or a pegylated form thereof.

The disclosure relates to a pharmaceutical composition comprising an FXR agonist and an IFN selected from the group consisting of IFN-α1a, IFN-α1b, and a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof. Preferably, the FXR agonist is selected from the group disclosed in Table 1. In one aspect, the FXR agonist is EYP001.

In an additional aspect, the disclosure relates to a kit comprising an IFN and an FXR agonist as a combined preparation for simultaneous, separate or sequential use, the IFN being selected from the group consisting of IFN-α1a, IFN-α1b, and a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof. Preferably, the FXR agonist is selected from the group disclosed in Table 1. In one aspect, the FXR agonist is EYP001.

In particular, the FXR agonist and the IFN are not administrated by the same route. For instance, the FXR agonist is administered by oral route whereas the IFN is administered by subcutaneous or intramuscular route. Alternatively, it can be considered to administer the FXR agonist and the IFN by the same administration route.

The pharmaceutical composition or kit as disclosed above is for use for the treatment of hepatitis B virus infection, in particular against the virus replication, for instance for the treatment of chronic hepatitis B. One aspect of the disclosure relates to
the use of said pharmaceutical composition or kit as disclosed above for the preparation of a medicament for the treatment of hepatitis B virus infection, in particular against the virus replication, for instance for the treatment of chronic hepatitis B;
an IFN selected from the group consisting of IFN-α1a, and IFN-α1b or a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof for use for the treatment of hepatitis B virus infection, in particular against the virus replication, for instance for the treatment of chronic hepatitis B, in combination with an FXR agonist, preferably selected from the group disclosed in Table 1, in particular EYP001;
an FXR agonist, preferably selected from the group disclosed in Table 1, in particular EYP001, for use for the treatment of hepatitis B virus infection, in particular against the virus replication, for instance for the treatment of chronic hepatitis B, in combination with an IFN selected from the group consisting of IFN-α1a, and IFN-α1b or a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof.

The present disclosure relates to a method for treating a subject infected by a hepatitis B virus, especially for treating a chronic hepatitis B in a patient, wherein the method comprises administering a therapeutic effective amount of an FXR agonist, preferably selected from the group disclosed in Table 1, in particular EYP001; and a therapeutic effective amount of an IFN selected from the group consisting of IFN-α1a, and IFN-α1b or a pegylated form thereof; IFN-β, preferably IFN-β1 such as IFN-β1a and IFN-β1b or a pegylated form thereof; IFN-γ1, especially IFN-γ1b, or a pegylated form thereof; and IFN-λ or IFN-λ or a pegylated form thereof, thereby decreasing the adverse effect of the IFN. In particular, the FXR agonist is administered at a therapeutic amount effective for decreasing the adverse effect of the IFN, especially the flu-like syndrome. Optionally, the FXR agonist is administered at a therapeutic amount effective for decreasing the adverse effect of the IFN, especially the flu-like syndrome, and for decreasing the replication of hepatitis B virus infection.

In a very specific embodiment, the present disclosure relates to a method for treating a subject infected by a hepatitis B virus, especially for treating a chronic hepatitis B in a patient, wherein the method comprises administering a therapeutic effective amount of EYP001; and administering a therapeutic effective amount of IFNα2a, IFNα2b or a pegylated form thereof;

wherein
EYP001 is administered at a therapeutic amount effective for decreasing the adverse effect of IFNα2a, IFNα2b or a pegylated form thereof, especially the flu-like syndrome; more preferably at a therapeutic amount effective for decreasing the adverse effect of the IFNα2a, IFNα2b or a pegylated form thereof and for decreasing the replication of HBV; more specifically, at a daily dose from 50 to 800 mg per adult per day, preferably from 100 to 600, still more preferably from 150 to 400 mg per adult per day or from 200 to 400 mg per adult per day; and for instance about 300 mg per adult per day; optionally administered once or twice a day, preferably orally; and
the IFNα2a, IFNα2b or a pegylated form thereof is administered by subcutaneous route once a week; for instance, at a dosage varying from 1 μg to 500 μg, preferably from 10 μg to 500 μg, more preferably from 100 μg to 250 μg, such as 100, 110, 120, 130, 140, 150, 160, 170, 180, 190 or 200 μg;
   thereby decreasing the adverse effect of the IFNα2a, IFNα2b or the pegylated form thereof. Optionally, the treatment lasts from 2-4 months up to 24 months, for instance between 2 and 24 months or between 2 and 12 months, e.g., 2, 3, 4, 5, 6, 7, 8, 9, 10, 11, 12, 13, 14, 15, 16, 17, 18, 19, 20, 21, 22, 23 or 24 months.

By decreasing HBV replication, it means that the HBV replication is decreased by at least 10 or 100 fold in comparison with the HBV replication in absence of EYP001. HBV replication can be assessed by determining the level of at least one among HBeAg levels, HBsAg levels, HBcrAg levels, pre-genomic RNA (HBV pgRNA) levels, pre-core RNA levels, relaxed circular DNA (HBV rcDNA) levels, HBV cccDNA levels or HBV DNA levels. For instance, the HBV replication can be assessed by determining the HBV DNA levels and this level is decreased by at least 10 or 100 fold in comparison with the HBV replication in absence of EYP001. Alternatively, HBV cccDNA level is decreased by at least 10, 15, 20, 25, 30, 35, 40, 45 or 50% in comparison with the absence of treatment.

In this embodiment, the present disclosure relates to a pharmaceutical composition comprising EYP001 for use for the treatment a subject infected by a hepatitis B virus, especially for use for treating a chronic hepatitis B, wherein the pharmaceutical composition is used in combination with IFNα2a, IFNα2b or a pegylated form thereof and EYP001 is to be administered at a therapeutic amount effective for decreasing the adverse effect of the IFNα2a, IFNα2b or the pegylated form thereof. It also relates to the use of a pharmaceutical composition comprising EYP001 for the manufacture of a medicament for use for the treatment a subject infected by a hepatitis B virus, especially for use for treating a chronic hepatitis B, wherein the pharmaceutical composition is used in combination with IFNα2a, IFNα2b or a pegylated form thereof and EYP001 is to be administered at a therapeutic amount effective for decreasing the adverse effect of the IFNα2a, IFNα2b or the pegylated form thereof. Preferably, the therapeutic amount to be administered is effective for decreasing the adverse effect of the IFNα2a, IFNα2b or the pegylated form thereof and for decreasing the replication of HBV. For instance, the daily dose of EYP001 is from 50 to 800 mg per adult per day, preferably from 100 to 600 mg per adult per day, still more preferably from 150 to 400 mg per adult per day or from 200 to 400 mg per adult per day,; and for instance about 300 mg per adult per day and it can be administered once or twice a day, preferably orally. Preferably, the IFNα2a, IFNα2b or a pegylated form thereof is to be administered by subcutaneous route once a week; for instance at a dosage varying from 1 μg to 500 μg, preferably from 10 μg to 500 μg, more preferably from 100 μg to 250 μg, such as 100, 110, 120, 130, 140, 150, 160, 170, 180, 190 or 200 μg. Optionally, the treatment lasts from 2-4 months up to 24 months, for instance between 2 and 24 months or between 2 and 12 months, e.g., 2, 3, 4, 5, 6, 7, 8, 9, 10, 11, 12, 13, 14, 15, 16, 17, 18, 19, 20, 21, 22, 23 or 24 months. More particularly, the EYP001 is administered as long as the treatment with the IFNα2a, IFNα2b or a pegylated form thereof is carried out.

The present disclosure further relates to a pharmaceutical composition or a kit as a combined preparation for simultaneous, separate or sequential use comprising an IFN-α and an FXR agonist for use for treating a disease selected from the group consisting of an infection by a virus chosen among hepatitis C virus (HCV), hepatitis D virus (HDV), herpes simplex virus (HSV), papillomavirus (HPV) (e.g., condylomata acuminate), varicella-zoster virus, cytomegalovirus (CMV) and rhinoviruses; a cancer, particularly a solid cancer or a hematopoietic cancer, preferably chosen among AIDS-related Kaposi's sarcoma, leukemia such as hairy-cell leukemia, chronic myeloid leukemia and non-Hodgkin's leukemia, lymphoma such as follicular lymphoma, cutaneous T-cell lymphoma and adult T-cell leukemia-lymphoma, carcinoid tumors, melanoma, multiple myeloma, renal cell carcinoma and neuroendocrine tumors; and other diseases such as age-related macular degeneration, angiomatous disease, Behçet's syndrome, thrombocythemia, polycythemia vera, agnogenic myeloid metaplasia, Churg-Strauss syndrome, inflammatory bowel disease and mycobacterial infection. It further relates to the use of an IFN-α and an FXR agonist for the preparation of a medicament for treating such diseases, to an IFN-α for use in combination with an FXR agonist for treating such diseases, to an FXR agonist for use in combination with an IFN-α for treating such diseases, and to a method for treating such diseases in a patient comprising administering a therapeutically effective amount of an FXR agonist and a therapeutically effective amount of an IFN-α, thereby decreasing the adverse effects resulting from a treatment with the IFN-α. The IFN-α can be selected from the group consisting of IFN-α1a, IFN-α1b, IFN-α2a and IFN-α2b or a pegylated form thereof. The FXR agonist can be selected from the group disclosed in Table 1, in particular EYP001. In a very specific aspect, the IFN-α is IFN-α2a or a pegylated form thereof and the FXR agonist is EYP001. More particularly, the FXR agonist is administered as long as the treatment with IFN-α is carried out. In particular, the FXR agonist is administered at a therapeutic amount effective for decreasing the adverse effect of the IFN-α, especially the flu-like syndrome. In one aspect, the FXR agonist is administered at a therapeutic amount effective for decreasing the adverse effect of the IFN-α, especially the flu-like syndrome, and for having a therapeutic effect on one of the diseases as defined above. For instance, the daily dose of EYP001 is from 50 to 800 mg per adult per day, preferably from 100 to 600 mg per adult per day, still more preferably from 150 to 400 mg per adult per day or from 200 to 400 mg per adult per day,; and for instance about 300 mg per adult per day and it can be administered once or twice a day, preferably orally. Preferably, the IFNα2a, IFNα2b or a pegylated form thereof is to be administered by subcutaneous route once a week; for instance at a dosage varying from 1 μg to 500 μg, preferably from 10 μg to 500 μg, more preferably from 100 μg to 250 μg, such as 100, 110, 120, 130, 140, 150, 160, 170, 180, 190 or 200 μg. Optionally, the treatment lasts from 2-4 months up to 24 months, for instance between 2 and 24 months or between 2 and 12 months, e.g., 2, 3, 4, 5, 6, 7, 8, 9, 10, 11, 12, 13, 14, 15, 16, 17, 18, 19, 20, 21, 22, 23 or 24 months.

The present disclosure further relates to a pharmaceutical composition or a kit as a combined preparation for simultaneous, separate or sequential use comprising an IFN-β and an FXR agonist for use for treating a disease selected from the group consisting of multiple sclerosis, Guillain-Barré syndrome, rheumatoid arthritis and a cancer, particularly a solid cancer or a hematopoietic cancer. It further relates to the use of an IFN-β and an

FXR agonist for the preparation of a medicament for treating such diseases, to an IFN-β for use in combination with an FXR agonist for treating such diseases, to an FXR agonist for use in combination with an IFN-β for treating such diseases, and to a method for treating such diseases in a patient comprising administering a therapeutically effective amount of an FXR agonist and a therapeutically effective amount of an IFN-β, thereby decreasing the adverse effects resulting from a treatment with the IFN-β. The IFN-β can be IFN-β1 such as IFN-β1a and IFN-β1b or a pegylated form thereof. The FXR agonist can be selected from the group disclosed in Table 1. In a very specific aspect, the FXR agonist is EYP001. More particularly, the FXR agonist is administered as long as the treatment with IFN-β is carried out. In particular, the FXR agonist is administered at a therapeutic amount effective for decreasing the adverse effect of the IFN-β, especially the flu-like syndrome. In one aspect, the FXR agonist is administered at a therapeutic amount effective for decreasing the adverse effect of the IFN-β, especially the flu-like syndrome, and for having a therapeutic effect on one of the diseases as defined above.

The present disclosure further relates to a pharmaceutical composition or a kit as a combined preparation for simultaneous, separate or sequential use comprising an IFN-γ and an FXR agonist for use for treating a disease selected from the group consisting of bacterial infections, in particular mycobacterial infections, fibrosis such as cryptogenic fibrosing alveolitis, leishmaniasis, osteoporosis and a cancer, particularly a solid cancer or a hematopoietic cancer. It further relates to the use of an IFN-γ and an FXR agonist for the preparation of a medicament for treating such diseases, to an IFN-γ for use in combination with an FXR agonist for treating such diseases, to an FXR agonist for use in combination with an IFN-γ for treating such diseases, and to a method for treating such diseases in a patient comprising administering a therapeutically effective amount of an FXR agonist and a therapeutically effective amount of an IFN-γ, thereby decreasing the adverse effects resulting from a treatment with the IFN-γ. The IFN-γ can be IFN-γ1, especially IFN-γ1b, or a pegylated form thereof. The FXR agonist can be selected from the group disclosed in Table 1. In a very specific aspect, the FXR agonist is EYP001. More particularly, the FXR agonist is administered as long as the treatment with IFN-γ is carried out. In particular, the FXR agonist is administered at a therapeutic amount effective for decreasing the adverse effect of the IFN-γ, especially the flu-like syndrome. In one aspect, the FXR agonist is administered at a therapeutic amount effective for decreasing the adverse effect of the IFN-γ, especially the flu-like syndrome, and for having a therapeutic effect on one of the diseases as defined above.

The present disclosure further relates to a pharmaceutical composition or a kit as a combined preparation for simultaneous, separate or sequential use comprising an IFN-λ and an FXR agonist for use for treating a disease selected from the group consisting of fibrosis (WO18115199) and hepatitis D virus infection (WO17143253). It further relates to the use of an IFN-λ and an FXR agonist for the preparation of a medicament for treating fibrosis or hepatitis D virus infection, to an IFN-λ for use in combination with an FXR agonist for treating fibrosis or hepatitis D virus infection, to an FXR agonist for use in combination with an IFN-λ for treating fibrosis or hepatitis D virus infection, and to a method for treating fibrosis or hepatitis D virus infection in a patient comprising administering a therapeutically effective amount of an FXR agonist and a therapeutically effective amount of an IFN-λ, thereby decreasing the adverse effects resulting from a treatment with the IFN-λ. The IFN-λ can be IFN-λ or a pegylated form thereof. The FXR agonist can be selected from the group disclosed in Table 1. In a very specific aspect, the FXR agonist is EYP001. More particularly, the FXR agonist is administered as long as the treatment with IFN-λ is carried out. In particular, the FXR agonist is administered at a therapeutic amount effective for decreasing the adverse effect of the IFN-λ, especially the flu-like syndrome. In one aspect, the FXR agonist is administered at a therapeutic amount effective for decreasing the adverse effect of the IFN-λ, especially the flu-like syndrome, and for having a therapeutic effect on fibrosis or hepatitis D virus infection.

IFNs can be used alone or in combination with other therapeutic agents. The other therapeutic agents can be for instance an antitumoral drug, an antiviral drug, an antibacterial agent, an anti-inflammatory agent, an immunosuppressive molecule. A non-exhaustive list of therapeutic agents that can be used in combination with IFNs includes tamoxifen; megestrol acetate; an anthracycline such as epirubicin, doxorubicin, daunorubicin, idarubicin, nemorubicin, pixantrone, sabarubicin and valrubicin; lonidamine; an antimetabolite such as 5-Fluorouracil (5-FU), 6-Mercaptopurine (6-MP), capecitabine

(Xeloda®), cytarabine (Ara-C®), floxuridine, fludarabine, gemcitabine (Gemzar®), hydroxycarbamide, methotrexate, pemetrexed (Alimta®), vinblastine; cisplatin, carboplatin or dicycloplatin; cytokine/hormone such as IL-2, TNF-α, octreotide; a nitrogen mustard alkylating agent such as cyclophosphamide or melphalan; retinoids such as acitretin; antiviral drugs such as ribavirin, taribavirin, simeprevir, sofosbuvir, zidovudine, lopinavir; antibiotics such as minocycline.

For instance, a specific combination of therapeutic agents can be selected in the non-exhausted list: IFN-γ+TNF-α+nitrogen mustard alkylating agent such as cyclophosphamide or melphalan; IFN-α+ribavirin; IFN-α+IL-2; IFN-α+Zidovudine; IFN-α+vinblastine; IFN-α+octreotide; IFN-α+TNF-α; IFN-β+minocycline; IFN-β+lopinavir+ritonavir; IFN-β+methylprednisolone; etc . . .

In a particular aspect, the FXR agonist, especially a FXR agonist of Table 1, and more particularly EYP001, and IFN-α or a pegylated form thereof can be used in combination with at least one additional active ingredient. Preferably, the additional active ingredient is an antiviral, more particularly an antiviral having an activity against HBV. In this context, the combination of FXR agonist and IFN is used for the treatment of HBV infection, in particular chronic HBV. In a preferred aspect, the at least one additional active ingredient is a polymerase inhibitor selected from the group consisting of L-nucleosides, deoxyguanosine analogs and nucleoside phosphonates. In a very specific aspect, the at least one additional active ingredient is selected from the group consisting of lamivudine, telbivudine, emtricitabine, entecavir, adefovir and tenofovir.

Further aspects and advantages of the present invention will be described in the following examples, which should be regarded as illustrative and not limiting.

### EXAMPLES

25 patients chronically infected with HBV underwent a 4-week treatment combining IFN (weekly sub-cutaneous injections of pegylated IFNα2a, PEG-IFN) with daily oral FXR agonist EYP001a or placebo. Overall 21 (84%) patients developed flu-like adverse events related to IFN: fever, weakness, muscle pain, headache, back or leg pain, bones or muscles aches, myalgia and fatigue. The frequency of flu-like AE was unexpectedly lower up to three times less when IFN treatment was combined with EYP001 (Table 2) with significant differences. Patients characteristics (Tables 3 and 4) did not differ across treatment arms and did not explain the reduction in IFN associated flu-like AE (Table 2). Summaries of patient characteristics and HBV infection parameters are provided in Tables 3 and 4, respectively.

TABLE 2
Frequencies of overall TEAE
(Treatment Emergent Adverse Events),
TEAE related to EYP001a,
TEAE related to IFN and Flu-like AEs.

| | | | | Flu-like | Non-Flu |
|---|---|---|---|---|---|
| | | EYP001a | PEG-IFN | Adverse | like |
| | TEAE | Related | Related | Event | TEAE |
| Treatment arm | (n) | TEAE (n) | TEAE (n) | n (%) | n (%) |
| EYP001a | 43 | 11 | 29 | 12 | 31 |
| (1 × 300 | | | | (17.6%)* | (40.3%) |
| mg/day) + INF | | | | | |
| (subjects n = 8) | | | | | |
| EYP001a | 47 | 12 | 36 | 18 | 29 |
| (2 × 150 | | | | (26.5%)* | (37.7%) |
| mg/day) + INF | | | | | |
| (subjects n = 9) | | | | | |
| Placebo + INF | 55 | 7 | 49 | 38 | 17 |
| (subjects n = 8) | | | | (55.9%) | (22.1%) |
| Total | 145 | 30 | 114 | 68 | 77 |
| | | | | (100.0%) | (100.0%) |

*p < 0.05 Chi-square statistic:

TABLE 3
Summary of patient characteristics at baseline

| | | EYP001a | | EYP001a | | | | | | | |
|---|---|---|---|---|---|---|---|---|---|---|---|
| | | (1 × 300 mg) + | | (2 × 150 mg) + | | EYP001a + | | Placebo + | | | |
| | | PEG-IFN | | PEG-IFN | | PEG-IFN | | PEG-IFN | | All | |
| Parameter | Statistic/ | (18 μg) | | (18 μg) | | Total | | (18 μg) | | Subjects | |
| (units) | stratum | (N = 8) | | (N = 9) | | (N = 17) | | (N = 8) | | (N = 25) | |
| Age (Years) | Mean (SD) | 41.6 | (11.0) | 38.6 | (9.5) | 40.0 | (10.0) | 37.9 | (9.9) | 39.3 | (9.8) |
| Gender n (%) | Female | 3 | (38%) | 5 | (56%) | 8 | (47%) | 2 | (25%) | 10 | (40%) |
| | Male | 5 | (63%) | 4 | (44%) | 9 | (53%) | 6 | (75%) | 15 | (60%) |
| Race n (%) | Asian | 2 | (25%) | 3 | (33%) | 5 | (29%) | 2 | (25%) | 7 | (28%) |
| | Black | 2 | (25%) | — | | 2 | (12%) | 1 | (13%) | 3 | (12%) |
| | White | 4 | (50%) | 6 | (67%) | 10 | (59%) | 5 | (63%) | 15 | (60%) |
| Height (cm) | Mean (SD) | 170.0 | (8.9) | 173.9 | (7.7) | 172.1 | (8.3) | 173.8 | (9.9) | 172.6 | (8.6) |
| Weight (kg) | Mean (SD) | 72.59 | (11.21) | 75.84 | (17.11) | 74.31 | (14.29) | 76.45 | (24.48) | 75.00 | (17.66) |
| BMI (kg/m²) | Mean (SD) | 25.04 | (2.18) | 24.78 | (3.89) | 24.90 | (3.11) | 25.05 | (6.82) | 24.95 | (4.47) |

TABLE 4
Summary of HBV infection parameters at baseline

| | EYP001a | EYP001a | | | |
|---|---|---|---|---|---|
| | (1 × 300 | (2 × 150 | EYP001a + | Placebo + | |
| | mg) + PEG- | mg) + PEG- | PEG-IFN | PEG- | |
| | IFN(18 μg) | IFN(18 μg) | Total | IFN(18 μg) | All Subjects |
| | (N = 8) | (N = 9) | (N = 17) | (N = 8) | (N = 25) |
| ALT (mean, SD) | 34.7 (38.6) | 27.3 (13.5) | 30.4 (26.2) | 32.2 (12.4) | 31.0 (22.6) |
| HBV DNA | 4.17 (1.79) | 4.26 (1.80) | 4.22 (1.74) | 4.87 (2.49) | 4.42 (1.98) |
| baseline mean | | | | | |
| log10 IU/mL (SD) | | | | | |
| HBsAg Igo10 | 3.8 (0.6) | 3.4 (1.1) | 3.6 (0.9) | 3.9 (0.8) | 3.7 (0.9) |
| IU/mL (SD) | | | | | |
| HBV Treatment naive | 5 (63%) | 8 (89%) | 13 (76%) | 3 (38%) | 16 (64%) |
| HBV Genotype A | 3 (38%) | 1 (11%) | 4 (24%) | 4 (50%) | 8 (32%) |
| HBV Genotype B | 1 (13%) | — | 1 (6%) | 2 (25%) | 3 (12%) |
| HBV Genotype C | 1 (13%) | 2 (22%) | 3 (18%) | — | 3 (12%) |
| HBV Genotype D | — | 1 (11%) | 1 (6%) | 1 (13%) | 2 (8%) |
| HBV Genotype E | 1 (13%) | — | 1 (6%) | — | 1 (4%) |
| HBeAg neg | 7 (88%) | 7 (78%) | 14 (82%) | 5 (63%) | 19 (76%) |
| HBeAg pos | | 1 (11%) | 1 (6%) | 1 (13%) | 2 (8%) |
| anti-HBeAg pos | 7 (88%) | 7 (78%) | 14 (82%) | 5 (63%) | 19 (76%) |

1-17. (canceled)

18. A method of decreasing adverse effects resulting from a treatment with an interferon (IFN) in a subject comprising administering EYP001 or a pharmaceutical composition thereof to the subject treated with IFN and experiencing adverse effects from said treatment.

19. The method according to claim 18, wherein the interferon is selected from the group consisting of IFN-α, IFN-β, IFN-γ, IFN-λ and pegylated forms thereof.

20. The method according to claim 18, wherein the adverse effects are the flu-like syndrome, fever, weakness, muscle pain, headache, back or leg pain, bones or muscles aches, myalgia, or fatigue.

21. The method according to claim 18, wherein the interferon is IFN-α or a pegylated form thereof.

22. The method according to claim 18, wherein the interferon is IFN-α2a and any pegylated form thereof.

23. The method according to claim 18, wherein the interferon is a pegylated IFN-α or a pegylated IFN-α2a.

24. The method according to claim 18, wherein the subject is infected by a hepatitis B virus or has chronic hepatitis B.

25. The method according to claim 18, wherein EYP001 is administered once or twice a day.

26. A kit comprising an IFN and EYP001, wherein the IFN is selected from the group consisting of IFN-α1a, IFN-α1b, and pegylated forms thereof; IFN-β, IFN-β1, IFN-β1a, IFN-β1b, or pegylated forms thereof; IFN-γ1, IFN-γ1b, or pegylated forms thereof; and IFN-λ or a pegylated form thereof.

27. A method of treating a subject having a hepatitis B virus infection comprising the administration of IFN and EYP001 to said subject, said IFN and EYP001 being administered separately, sequentially, simultaneously or as a combined composition.

28. The method according to claim 27, wherein EYP001 is administered once or twice a day.

29. A method of treating a subject having a disease comprising the administration of a IFN-α and EYP001 to said subject, said IFN-α and EYP001 being administered separately, sequentially, simultaneously or as a combined composition and said disease being selected from the group consisting of:
an infection by a virus selected from the group consisting of hepatitis C virus (HCV), hepatitis D virus (HDV), herpes simplex virus (HSV), papillomavirus (HPV), varicella-zoster virus, cytomegalovirus (CMV) and rhinoviruses;
a cancer, a solid cancer, a hematopoietic cancer, AIDS-related Kaposi's sarcoma, leukemia, hairy-cell leukemia, chronic myeloid leukemia, non-Hodgkin's leukemia, lymphoma, follicular lymphoma, cutaneous T-cell lymphoma, adult T-cell leukemia-lymphoma, carcinoid tumors, melanoma, multiple myeloma, renal cell carcinoma, and neuroendocrine tumors; and
age-related macular degeneration, angiomatous disease, Behçet's syndrome, thrombocythemia, polycythemia vera, agnogenic myeloid metaplasia, Churg-Strauss syndrome, inflammatory bowel disease and mycobacterial infection.

30. The method according to claim 29, wherein the IFN-α is IFN-α1 IFN-α2, IFN-α1a, IFN-α1b, IFN-α2a, IFN-α2b or pegylated forms thereof.

31. The method according to claim 29, wherein EYP001 is administered once or twice a day.

32. A method of treating a subject having a disease comprising the administration of a pharmaceutical composition or kit according to claim 26 to said subject, said pharmaceutical composition or kit comprising an IFN-β and EYP001 and said disease being selected from the group consisting of multiple sclerosis, Guillain-Barré syndrome, rheumatoid arthritis, a cancer, a solid cancer and a hematopoietic cancer.

33. The method according to claim 32, wherein the IFN-β is IFN-β1, IFN-β1a, IFN-β1b or pegylated forms thereof.

34. The method according to claim 32, wherein EYP001 is administered once or twice a day.

35. A method of treating a subject having a disease comprising the administration of a pharmaceutical composition or kit according to claim 26 to said subject, said pharmaceutical composition or kit comprising an IFN-γ and EYP001 and said disease being selected from the group consisting of bacterial infections, mycobacterial infections, fibrosis, cryptogenic fibrosing alveolitis, leishmaniasis, osteoporosis, a cancer, a solid cancer and a hematopoietic cancer.

36. The method according to claim 35, wherein EYP001 is administered once or twice a day.

37. A method of treating a subject having a disease comprising the administration of a pharmaceutical composition or kit according to claim 26 to said subject, said pharmaceutical composition or kit comprising an IFN-λ and EYP001 and said disease being selected from the group consisting of fibrosis and a hepatitis D virus infection.

38. The method according to claim 37, wherein EYP001 is administered once or twice a day.
